(19) United States
(12) Patent Application Publication
(10) Pub. No.: US 2003/0032017 A1
(43) Pub. Date: Feb. 13, 2003

(54) Quantification of low molecular weight and low abundance proteins using high resolution two-dimensional electrophoresis and mass spectrometry

(75) Inventors: Norman G. Anderson, Rockville, MD (US); Madhu Mondal, Rockville, MD (US); Rembert Pieper, Washington, DC (US)

Correspondence Address: Dean H. Nakamura, Roylance Abrams Berdo & Goodman, 1300 19th Street, N.W., Washington, DC, 20036, US

(73) Assignee: Large Scale Proteomics, Corp.

(21) Appl. No.: 09/921,004

(22) Filed: Aug. 3, 2001

(51) Int. Cl. C12Q 1/68

(52) U.S. Cl. 435/6

(57) ABSTRACT

Methods are disclosed comprising specific technologies including a system for routinely concentrating proteins from human urine ranging down to approximately 2.5 kDa automated systems for immunosubtraction of major proteins form urine and plasma to reveal minor ones, and systems for routinely fractionating protein mixtures on the basis of native molecular weight, isoelectric point that are applicable to a range of human body fluid proteins, particularly those found in urine.

### FIELD OF INVENTION

Generally, this invention relates to methods of determining useful markers from body fluids and tissues using a logical and systematic approach comprising high-resolution chromatographic techniques. Specifically the invention relates to the discovery of low molecular weight and low abundance protein components that comprise urine. Using two dimensional electrophoresis (2DE) coupled with affinity, protein concentration methods, fractionation methods and mass spectrometry, spots are visualized in patterns which are subsequently used to develop a urinary proteome that can be correlated to various physiological conditions.

### BACKGROUND

Proteins present in mammalian body fluids such as whole blood, serum, plasma, cerebrospinal fluids, tears, sweat, sputum, saliva, urine and tissues are useful as indicators of certain disease states. Thus, methods for identifying and quantifying various proteins in clinical samples can provide clinicians with a great deal of information leading to the diagnosis of a variety of diseases. Further, such methods can also allow for automation culminating in high-throughput analysis of samples and simultaneous multiple analyte detection (e.g., via microarrays).

Investigations using non-quantitative or semi-quantitative 2DE have been to correlate protein expression with physiological state (Kanitz et al., Toxicol Methods (1997) 7(1):27-41; Schmid et al., Electrophoresis (1995) 16:1961-68; Tracy et al., Clin Chem (1982) 28:915-9; Rasmussen et al., J Urol (1996)155(6):2113-9; Rasmussen et al., Electrophoresis (1998) 19:818-25), and to develop databases of the protein composition of various tissues including liver (Wirth et al., Electrophoresis (1995) 16:1946-60), brain (Comings et al., Clin Chem (1982) 28:782-9), heart Corbett et al., Electrophoresis (1995) 16:1524-29), keratinocytes (Celis et al., Electrophoresis (1994) 15:1349-58), and blood proteins (Hughes et al., Electrophoresis (1992) 13:707-14), among others. However, focused technological development, required man-hours and levels of funding required to create, maintain and continually expand such databases are lacking in this area, and thus few clinically relevant novel protein disease markers have emerged from such investigations.

A number of techniques involving the analysis of proteins found in urine are known. These range from “dipstick” chemistry methods which simply indicate the presence or absence of proteins (i.e., mostly albumin) to relatively complex methods involving the separation, identification, and quantification of proteins which may be present at very low concentrations (e.g., immunological assays and creatine ratio analysis). For example, quantitative determination of urinary microalbumin may be carried out by RIA (radioimmunoassay) and immunoprecipitation (U.S. Pat. No. 5,246,835).

The amount and type of protein excreted in urine is controlled ultimately by the kidneys and function of the glomeruli. The glomeruli of the kidney behave as ultrafilters for the plasma proteins. The degree to which individual proteins are normally filtered through the glomerular membrane is a function of both their molecular weight and ionic charge, as well as their plasma concentration. In general, transport of protein molecules through the membrane progressively diminishes as protein size increases. Normally, high molecular weight proteins, such as IgM (MW 900,000) do not appear in glomerular filtrate except in trace amounts. Relatively small yet significant amounts of albumin (MW 66,000) are passed into the filtrate as a result of its high plasma concentration and relatively low molecular weight. Proteins of MW 15,000 to 40,000 filter more readily but in lesser quantities because of their low plasma concentrations. In addition, the proportions of individual proteins excreted in the urine depend on the extent of their reabsorption by the renal tubules; albumin represents approximately 60% of the total proteins excreted because it is not completely removed from the filtrate by tubular cells. The low molecular weight proteins are actively reabsorbed from the filtrate and catabolized in the proximal tubule.

Very little of the total urine protein normally excreted consists of these small proteins. Only a small amount of protein is excreted normally (20-150 mg/dl), and most of it is albumin, another important constituent being Tamm-Horsfall protein, probably secreted by the distal tubules.

Differential diagnosis of renal and other diseases, including their prognosis, is aided to a large degree on the evaluation of selectivity of the glomerular membrane. For example, patients afflicted with kidney or renal disorders can excrete urine containing relatively high amounts of albumin and other serum proteins typically not found at such concentrations in the urine of healthy individuals (e.g., >150 mg/day). Moreover, the urine of patients presenting certain cancers, such as myeloma patients, is known to contain specific proteins (e.g., free light chain gamma globulins or Bence-Jones proteins). Accordingly, techniques for identifying and quantifying these and other protein components of clinical significance from urine samples can provide indicators of abnormal conditions such as glomerulonephritis, acute nephritis as well as the presence of select cancers in patients.

While qualitative methods exist for measuring primarily albumin (e.g., the dipstick method) or, in the alternative, all urinary proteins (urine sulfosalicylic acid test), these methods do not have the required resolution necessary to identify selective or specific markers (e.g., the dipstick method cannot detect Bench-Jones proteins).

It is estimated that serum and urine may each contain more than 5,000 different proteins, ranging in concentration from up to 40 g/L, or less, of serum albumin down to nanogram/L concentration of hormones and other trace proteins. No single technique has the resolution and dynamic range required to resolve such mixtures. High resolution two-dimensional electrophoresis can resolve 1,000-2,000 proteins within a concentration dynamic range window of approximately 1,000:1, but cannot resolve that many in serum because a variety of very high abundance proteins are present. Likewise, the abundance of albumin and Tamm-Horsfall protein in such fluid can also greatly effect the dynamic range of the method. Greater quantification and precision involving separation of the urine protein components using molecular affinity/selectivity, electrophoresis and other chromatographic techniques followed by detecting the separated proteins can afford such resolution.

Accordingly, methods are provided to quantify natively low molecular weight urinary proteins in clinical samples to detect and to identify, in a clinical sample, components that are expressed in low abundance and ultimately use such components as disease marker. Further, uses are envisaged where such methods provide comparisons of urinary proteins between healthy and abnormal individuals as well as individuals exposed to drugs, toxins and other environmental pressures to identify responder proteins modulated by such physiological stresses.

### SUMMARY OF THE INVENTION

The instant invention relates to a method of detecting and quantifying low molecular weight protein and/or peptide components in a biological sample, particularly in urine. The method comprises a number of steps, amenable to automation, that include, but are not limited to, concentrating biological fluid; fractionating the concentrated material collected; separating the constituents of the fraction of interest and components of the original fluid. In a related protein and peptide identification is accomplished by mass spectrometry, including time of flight mass spectrometry. Such a method is envisaged to have use as a means for determining sequence as well as molecular weight to define fluid proteins and peptides.

The instant invention also relates to the generation of cognizable patterns as a means of analyzing the presence or absence of low molecular protein and/or peptide components comprising a biological fluid. In a related aspect, these patterns can be correlated with physiological state. Further, while the focus of the method centers on urinary proteins, the method is also useful in detecting proteins and/or peptides from biological fluids that include, but are not limited to, blood, cerebral spinal fluid, sputum, feces, tissues and sweat.

The method disclosed in the instant invention envisages the use of means to concentrate the components of a biological fluid, especially in view of the level of dilution of proteins and/or peptides in fluids such as urine. Such concentrating means includes, but is not limited to, size exclusion chromatography, reverse phase chromatography, hydrodynamic shear force (e.g., centrifugation), dialysis, and lyophilization. In a related aspect, the various concentration means may be combined. Further, such means may be reiterated as pre and post steps to dialysis, centrifugation and/or lyophilization, including addition of volatile salts such as ammonium bicarbonate. In a related aspect, conditions such as, but not limited to, for example, pH, mesh size, flow rates and stationary phase media selection can be modified to select for specific low molecular weight patterns.

The invention discloses the use of protease inhibitor in the body fluid during sample collection, to include, but not limited to, such inhibitors as antipain-HCl, bestatin, chymostatin, E-64, EDTA, leupeptin, PMSF, pepstatin and phosphoramidon.

Further, the method envisages the use of elution from an affinity matrix as a means of fractionating the concentrated materials. The matrices can comprise a column. Such columns may contain immunologic and non-immunologic affinity materials such as but not limited to the following: monoclonal and polyclonal antibodies, protein A, protein G, haptoglobin, arginine, benzamidine, glutathione, Cibachron blue, calmodulin, gelatin, heparin, lysine, lectins, Procion Red HE-3B, nucleic acids and metal affinity media. Moreover, such materials can include reverse phase matrices.

In a related aspect, the immunologic affinity materials may be directed, but not limited thereby, to albumin, tranferrin, a α1antitrypsin, α2macroglobulin, α1acid glycoprotein, C3, Tamm-Horsfall protein, hemopexin, α2HS glycoprotein, α1antichymotrypsin, Gc globulin and ceruloplasmin. In a further related aspect, the non-immunologic affinity materials may be directed, but not limited thereby, to serine proteases, glutathione S-transferases, glutathione-dependent proteins, enzymes requiring NAD+ and NADP+, albumin, coagulation factor, interferon, APTases, prokinases, phosodiesterases, neurotransmitters, fibronectin, growth factors, coagulation proteins, steroid receptors, plasminogen activator, hydrogenases and most other enzymes requiring adenyl-containing cofactors, binding to specific sugar on glycosylated proteins, DNA-binding proteins and serum proteins.

The method of the instant invention also relates to the use of separating means such as, but not limited to, two-dimensional electrophoresis (2DE) and zonal sedimentation centrifugation on density gradients. In a related aspect, the 2DE comprises the use of native isoelectric focusing to maintain subunit/complex association.

Another aspect of the instant invention envisages the generation of images of protein/peptide patterns from data collected from the analysis of body fluids, particularly from urine. These images can be manipulated to provide linkages to annotations. Such annotations may comprise, for example, information concerning patients, nucleic acid or amino acid sequence data, antibody selection, physicochemical protein data, protein abundance data and synthesis correlation data between modulation of said protein abundance and physiological state. In a related aspect, these images may be formed through an image data storing means, where the image data is being produced from images of stationary phases such as stained polyacrylamide gels and detectable regions of microarray surfaces. Such data is stored in a storage means, where such an image is displayed on a display means. Such a display means is envisaged to be adapted to display the image and pattern based on the stored image data.

In a further related aspect, the patterns generated can be selected by a pattern selecting means for selecting graphic data corresponding to patterns for defining regions of interest from among graphic data comprising stationary phase pattern data stored in a graphic data storing means. Further, the pattern selecting means is constituted so as to select predetermined graphic data from among the graphic data stored in the graphic storing means based on coordinate data specified by a cursor means displayed and moveable on the display means.

In a further aspect, the instant invention relates to the analysis of low molecular weight proteins in body fluids, such as urine, which low molecular weight proteins can be used as an indicator of tissue damage.

These and other advantages associated with the present invention and a more detailed explanation of preferred embodiments are described below and should be taken in combination with the following drawings.

### BRIEF DESCRIPTION OF THE DRAWINGS

FIG. 1 depicts a map of separated human plasma proteins.

FIGS. 2A-2C depict gel filtration scans of plasma and urine. FIG. 2C provides molecular weight standards.

FIG. 3 is a histogram of urinary proteins.

### DETAILED DESCRIPTION OF THE INVENTION

As used herein, the term “deflecting”, including grammatical variations thereof, refers to turn aside especially from a straight course or fixed direction.

As used herein, the term “cognizable”, including grammatical variations thereof, refers to as capable of being known.

As used herein, the term “body fluid”, including grammatical variations thereof, refers to liquid components of living organisms. For example, blood, lymph, serum and urine are body fluids. Tissues which have been homogenized or otherwise treated so that fluid is extracted therefrom are also considered body fluids.

As used herein, the term “stationary phase”, including grammatical variations thereof, refers to the inert matrix that allows for percolation of a mobile phase. For example, polyacrylamide gel of a 2DE system is a stationary phase.

As used herein, the term “lyophilized”, including grammatical variations thereof, refers to the creation of a stable preparation of a biological substance, such as blood plasma or serum, by rapid freezing and dehydration of the frozen product under high vacuum.

As used herein, the term “hydrodynamic shear”, including grammatical variations thereof, refers to the motion of fluids and the forces acting on solid bodies immersed in fluids and in motion relative to them.

As used herein, the term “native”, including grammatical variations thereof, refers to a substance found in nature especially in an unadulterated form. In contrast, “denatured” applies to exposure of proteins to substances such as detergents and/or nucleic acids to chaotropic agents such as formamide, that causes an alteration of the naturally occurring form.

As used herein, the term “linkage”, including grammatical variations thereof, refers to an identifier attached to an element (as an index term) in a system to indicate or to permit connection with other similarly identified elements.

As used herein, the term “annotation”, including grammatical variations thereof, refers to a note added by way of comment or explanation.

As used herein, the term “cursor”, including grammatical variations thereof, refers to a visual cue (e.g., flashing rectangle) on a video display that indicates position (e.g., as for data entry).

The human genome is estimated to contain approximately 35,000 genes that yield, counting the products of alternative splicing, posttranslational modifications, and proteolytic cleavages, a very much larger number of functional proteins. Many of these proteins are believed to be potential markers for human disease including cancer. Given such a large number of proteins, the wide dynamic range of their expression, and given the comparably large number of different human diseases, it is evident that it is infeasible to test every human protein against every disease state. Hence rational methods and means must be found for picking, among this vast array, the most likely candidates for further experimental and clinical studies.

This invention is concerned in part with methods for discovering tissue-specific or disease-specific proteins that may leak from the tissue into plasma (histemia) and which may the also appear in the urine (histuria). It is well known that injured tissues undergo a series of changes injury starting with swelling (oedema), followed by loss of salts, metabolites, and finally by the leakage of proteins. Interest centers initially on whether such leakage is a general phenomenon, whether proteins tending to leak might have any common properties that may facilitate their isolation, whether those properties are shared by known disease markers appearing in plasma or urine, and whether interesting and useful active factors have actually been found, especially in urine.

For urine, an additional factor of the filtration characteristics of the kidney glomeruli, and the physiology of the kidney tubules must be considered. A volume equivalent to the entire plasma volume is filtered through the kidney approximately every 20 minutes, hence 72 times the plasma volume is filtered very 24 hours. It is evident, therefore, that low molecular weight components of plasma will be rapidly removed, and that these are better sought in urine than in plasma, providing that they are not removed by the kidney tubules.

High-resolution two-dimensional electrophoresis (2DE) resolves up to several thousand different proteins in a single gel, and provides a global method for resolving complex mixtures. 2DE analyses are done under denaturing conditions, and reveal the isoelectric points and masses of protein subunits in di- or multimeric proteins, and the same parameters for proteins not natively composed of subunits. Hence one cannot infer from 2DE mass measurements the native mass of an individual protein.

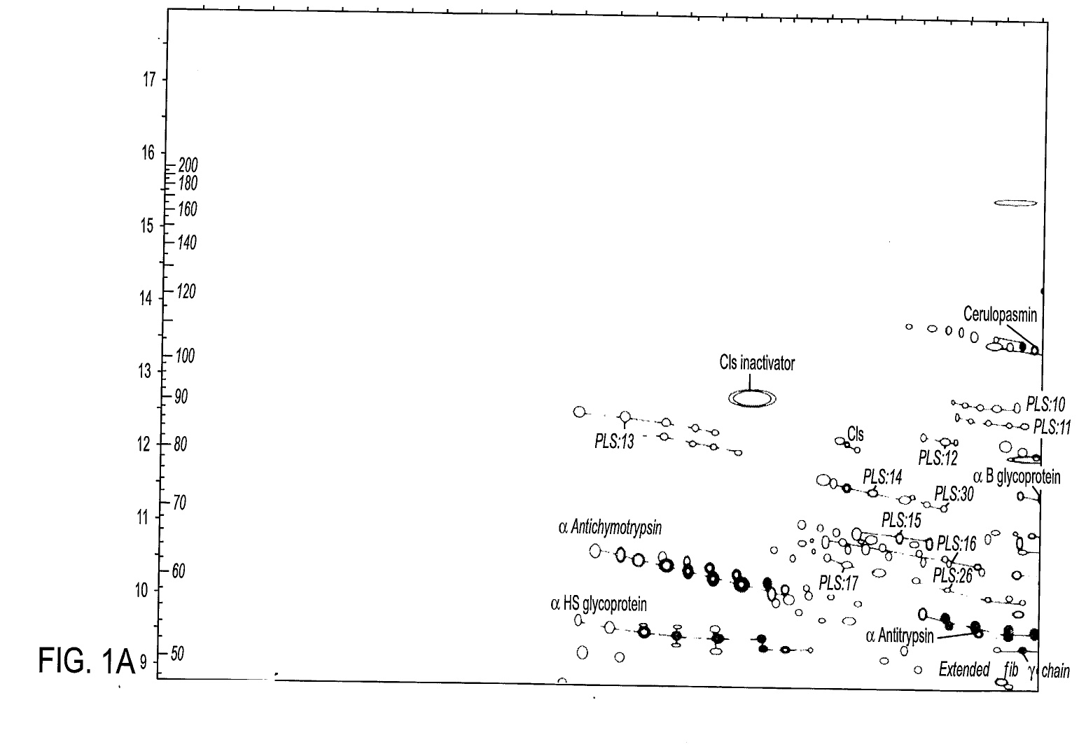

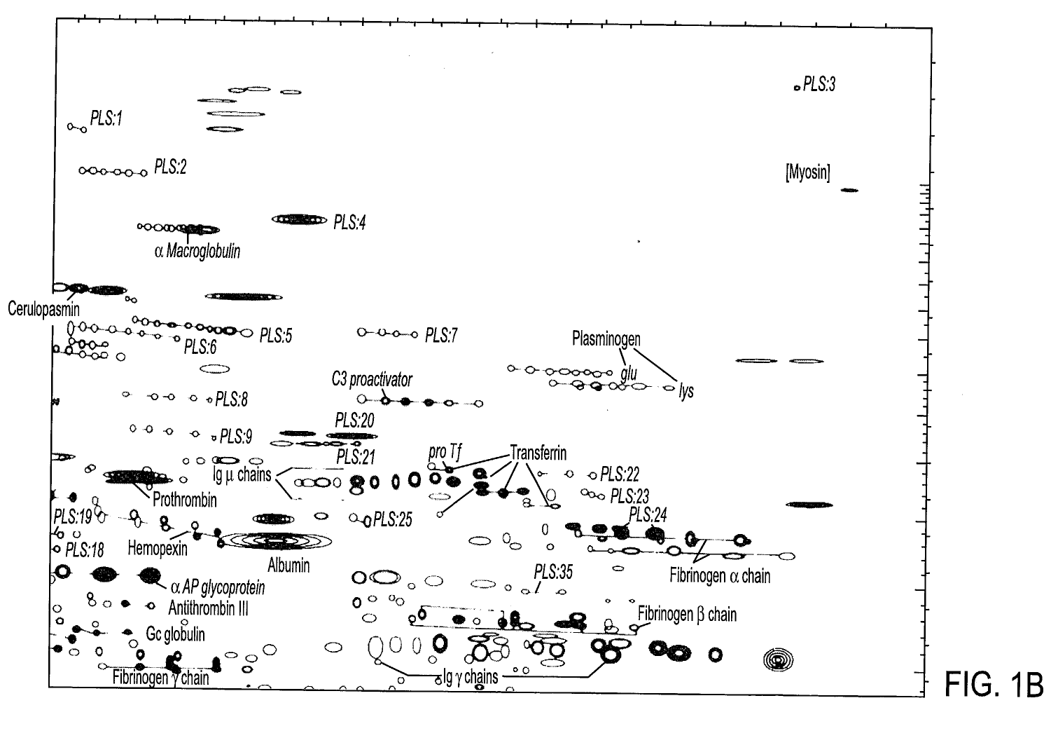

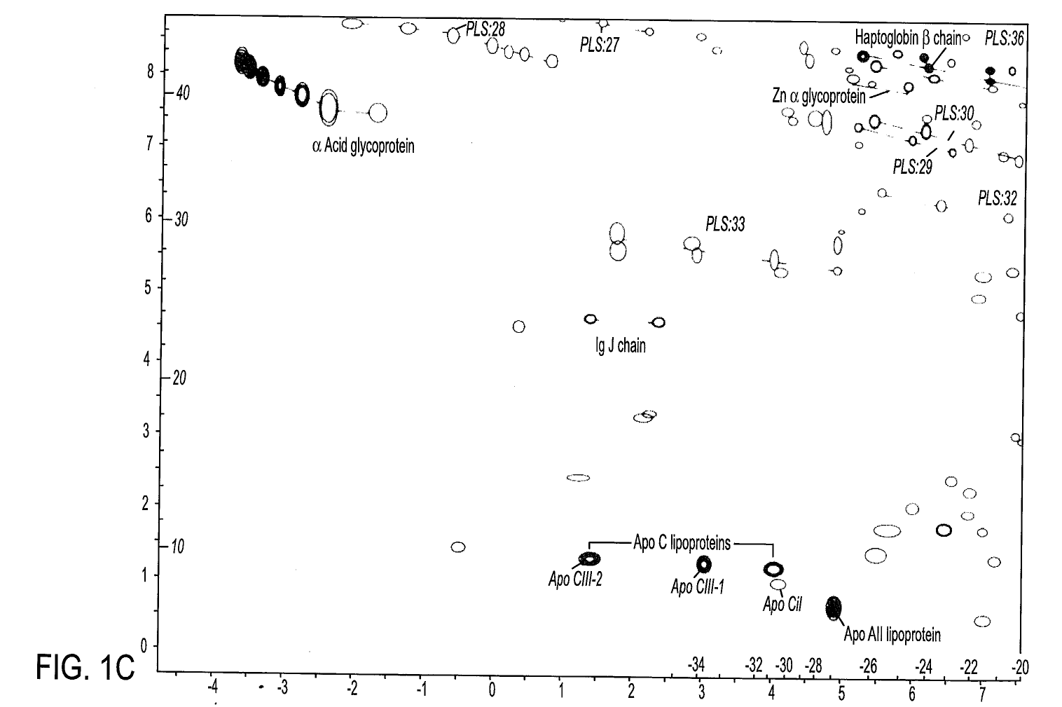

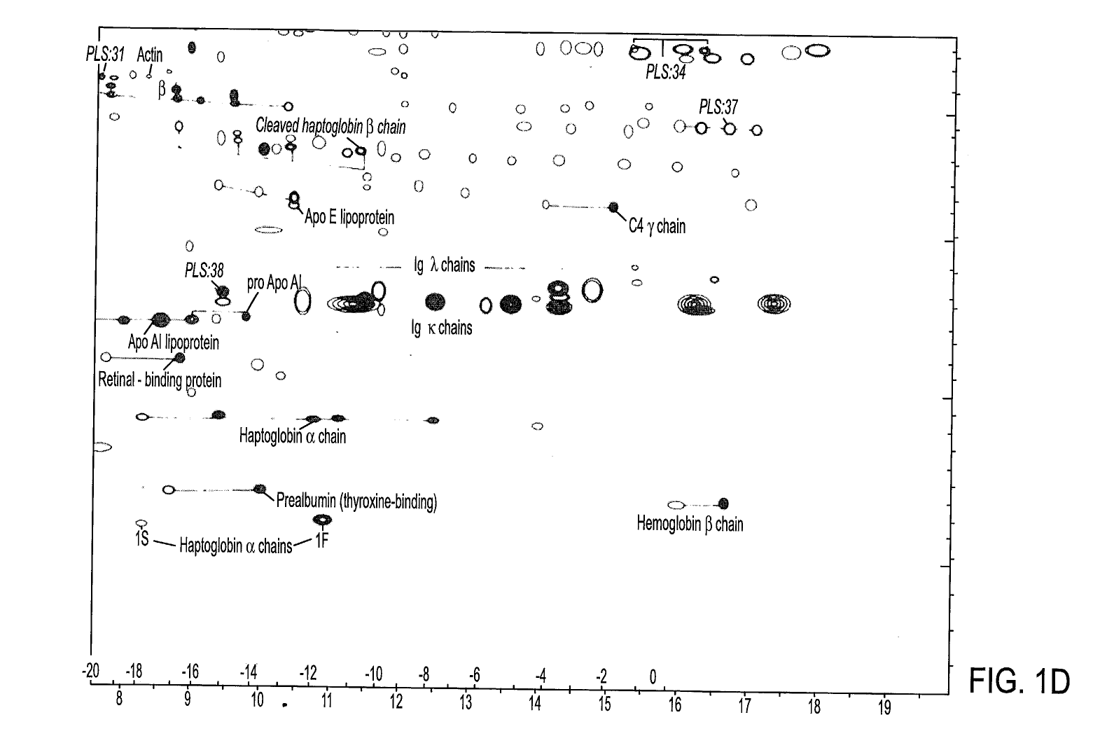

As shown in FIG. 1, 2DE analyses of human serum shows a very large number of proteins, and attempts have been made to use this technology to find new markers in human serum or plasma. Known proteins in human plasma exist in a very wide dynamic range, covering over ten orders of magnitude. Unfortunately, most useful markers appear in serum or plasma in the microgram per liter concentration range, making it difficult or impossible to find these by the straightforward analysis of serum or plasma from patient samples without extensive pre-fractionation done on very volumes of starting material. Methods have therefore been sought for finding potential marker proteins in tissues where they would be expected to be in much higher concentrations.

Most useful markers, for example those used to detect damage to the heart or liver, have been developed by looking for proteins, usually enzymes, that are unique to these organs. Alternatively, variants of well-known proteins known to be present in relatively large quantities have been assayed. The advantage of enzymes was that activities could be measured in very low concentrations, however with the development of very sensitive immunoassays very low abundances of minor proteins such as peptide hormones can be measured in serum in picogram per liter concentrations. Given such sensitive assays, the question then becomes one of finding new candidate tissue marker proteins that can be assayed in either blood or urine. Many of these may not be known enzymes, or have any enzyme activity at all.

One of the most difficult problems facing the Emergency Room physician today is the triage of patients with chest pain. The admission of patients with a low probability of acute coronary artery disease often leads to excessive hospital costs. Conversely, technologies and strategies that discharge too liberally may lead to misdiagnoses. Inappropriate discharge of ER patients who actually have an acute myocardial infarction (AMI) has been estimated to occur in 2-5% of patients, and is the single most common cause of malpractice lawsuits against ER physicians today.

While the present tests used to detect and classify AMI are useful, there is a growing awareness that better tests are required. Those in present use have been empirically discovered, and antedate the discovery technology described here. These is therefore an urgent need for new tests which are more sensitive, which provide an estimation of the extent of the infarction, which can be used to evaluate therapy on an ongoing basis, and which are predictive of the future course of the disease.

It is of interest to ask whether markers of human disease are generally above or below the glomerular filtration cut off point. The molecular weights of proteins that have been studied as markers of tissue injury is shown in Table 1. This table suggests that if one aims to find new markers of tissue damage and leakage, one would seek them among proteins having masses below 55 kD. The most widely used assays for heart damage are myosin, troponin, and creatine kinase, all of which have molecular weights below 55 kD.

Gel filtration, centrifugal membrane filtration, and differential high-speed centrifugation are well known methods for fractionating protein on the basis of mass.

Leakage proteins most used clinically include the measurement of myoglobin, treponins, and creatine kinase into the blood stream after heart attacks, and the appearance of transaminases in the blood after toxic injury to the liver. A list of proteins that have been measured clinically in serum is shown in Table 1. These have been examined under experimentally under a variety of conditions. The majority of these have molecular masses below 57,000 Daltons. This suggests but does not prove that injury to cell plasma membranes involves a gradual increase in permeability, and that the size distribution of proteins leaked may indicate the extent of disease or injury, with small ones appearing first, followed by larger ones as disease or injury is found to be more extensive, ending is tissue necrosis.

It is an objective of the present invention to provide a method and apparatus for discovering substances present in normal cells and tissues that are small enough to leak out of injured or diseased cells or tissues into plasma and/or urine, and can be there detected.

It is a further objective to recover that fraction of cells which are natively soluble and are commonly termed the cytosol from normal human tissues, to isolate those native proteins having relatively small molecular masses using biophysical means, to compare said protein fraction isolated from different human organs by high-resolution two-dimensional electrophoresis, and to discover candidate leakage proteins.

It is a further objective to discover, by comparative image analysis those proteins that are enriched in one or a few cell types or organs relative to others and designate them as candidate markers.

It is an additional objective to use candidate markers to prepare antibodies against these markers.

A still further objective is to use these antibodies to develop specific immunological tests for clinical evaluation as diagnostic indicators of disease.

An additional objective is to isolate and characterize by mass spectrometry or amino acid sequencing candidate marker proteins.

A further objective is to use sequence data to identify the gene or genes producing the candidate marker.

A still further objective is to identify candidate markers that are not tissue or organ specific, but which are absent from normal plasma or urine, and which could serve as general or global indicators of disease or injury.

It is yet another objective of the present invention to find markers that are present in a limited but defined set of tissues or organs, for example those derived from one germ layer.

In the method of this invention cells or tissues are ground or homogenized to break some fraction of the cells present. The homogenate, is then centrifuged to sediment particulate matter and the supernatant, termed the cytosol, recovered. This cytosol is then fractionated by gel filtration into at least two fractions differing in native molecular mass. Both fractions, on analysis by denaturing high-resolution two-dimensional electrophoresis exhibit low molecular weight proteins. However the high molecular weight proteins are absent, or present in very low abundance, in the natively lower molecular weigh fraction.

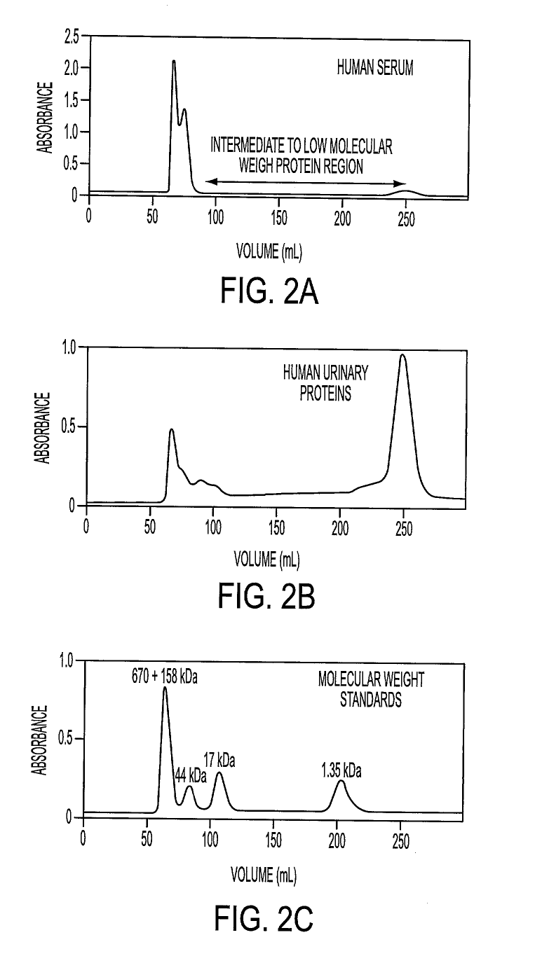

In the 2DE pattern shown in FIG. 1, electrophoresis in both dimensions (isoelectric focusing and SDS electrophoresis) is run under denaturing conditions that dissociate dimeric or multimeric proteins into subunits. Examination of this figure might suggest that the proteins present in plasma exist over a wide range of sizes extending from several thousand down to the lower limits of resolution of this system that are around 10-15 kiloDaltons. This, however, is not actually the case in the blood stream, and gel filtration analysis of human serum, shown in FIG. 2, shows that there are almost no proteins present below approximately 55 kD. This, as expected, matches the cutoff of the glomerular filtration system of the kidney. Normal native serum or plasma therefore has almost no proteins present below this cutoff figure.

This means that using gel filtration, and/or differential centrifugation it is feasible to remove the majority of the smaller proteins present in human urine.

Cells and tissues have large numbers of proteins in the mass range below 55 Kd from which to chose. 2DE does not indicate directly the native mass of the proteins resolved, and hence 2DE can be misleading. This question has been examined by analyzing, by first fractionating human heart cytosol using gel filtration. It is evident that heart cytosol contains a large fraction of proteins below circa 50 kD. The proteins of the starting mixture, and the fractionated >30 kD and <30 kD proteins were then analyzed by DE. If there are no native proteins smaller than ˜55 kD, there should be no proteins present in the <30 kD fraction analyzed. The natively >30 kD protein fraction contains proteins that, when denatured, covers the entire mass range resolved. Many proteins are present that are natively small, and, as expected appear similarly small on denatured 2DE patterns. Note that the cutoff of gel filtration columns is not sharp, and further research is required to optimize gel filtration fractionation of plasma and urinary proteins. It is concluded from these studies and additional research that, unlike serum or plasma, cells and tissues have a large fraction of proteins that are below the cutoff for the kidney, and are therefore in the range of size range of known marker proteins.

Almost any of the abundant proteins in the range below 30-50 kD could, in theory, serve as non-specific injury markers. The most useful markers, however, would be those that are cell, tissue or disease specific. 2DE has been used to survey brain tissues and proteins relatively specific for brain discovered, demonstrating that 2DE can be used to discover new markers.

TABLE 1
Markers of Tissue Damage

| PROTEIN | | MW | pI | ORGAN | |
|---|---|---|---|---|---|
| 1. | Myoglobin | 17,052.6 | 7.29 | Heart | |
| 2. | Creatine Kinase B | 42,644.2 | 5.34 | Heart | |
| 3. | Creatine Kinase M | 43,101.1 | 6.7 | Heart | |
| 4. | Cardiac Troponin T | 25,038.5 | 5.45 | Heart | |
| 5. | Cardiac Troponin I | 23,900 | | Heart | |
| 6. | Beta- | 19,329.1 | 5.37 | Heart | |
| | hydroxybutyrate | | | | |
| | Dehydrogenase | | | | |
| 7. | C-reactive Protein | 25,038.5 | 5.45 | Heart | |
| 8. | Heart Fatty Acid | 14,726.0 | 6.34 | Heart | |
| | Binding Protein | | | | |
| 9. | C4 Aldolase | 39,324 | 6.46 | Brain | |
| 10. | Alkaline | 57,293 | 6.29 | Liver | |
| | Phosphatase | | | | |
| 11. | Gamma Glutamyl | 61,382 | 6.65 | Liver | Microsomal? |
| | Transpeptidase | | | | |
| 12. | Glutamic Oxalo- | 47,475 | 9.14 | Liver | Mitochondrial |
| | acetic | | | | Enzyme |
| | Transaminase | | | | |
| | (Aspartate | | | | |
| | transaminase | | | | |
| 13. | Glutamic Oxalo- | 46,116 | 6.57 | Liver | Cytoplasmic |
| | acetic | | | | |
| | Transaminase | | | | |
| | (Aspartate | | | | |
| | transaminase | | | | |
| 14. | Glutamic pyruvic | 54,440 | 6.8 | Liver | Cytosolic |
| | transaminase | | | | |
| 15. | 5′ Nucleotidase | 64,969 | 5.75 | Placenta | Cytosol? |
| | | | | | See ref. |
| 16. | Acid Phosphatase | 17,911 | 6.35 | Adi- | |
| | | 17,846 | 6.48 | pocytes | |
| 17. | Alanine | 54,439.6 | 6.81 | | |
| | Aminotransferase, | 43,009.9 | 8.61 | | |
| | Alanine | | | | |
| | Transaminase | | | | |
| 18. | Enolase | 47,037.8 | 6.99 | Non- | |
| | | | | neuronal | |
| | | 46,855.7 | 7.73 | Skeletal | |
| | | | | muscle | |
| | | | | Neuronal | |
| | | 47,154.4 | 4.94 | | |
| 19. | Amylase | 57,767.8 | 6.47 | Salivary | |
| 20. | Cholinesterase | | | | |
| 21. | Chymotrypsin | 27,869.9 | 6.79 | B | |
| | | 9 | | precursor | |
| 22. | Glutamate | 61,397.8 | 7.66 | Mito- | |
| | Dehydrogenase | 7 | | precursor | |
| | | | 8.63 | Mito- | |
| | | | | precursor | |
| | | 61,434.0 | | | |
| | | 0 | | | |
| 23. | Isocitrate | 39,591.6 | 6.46 | Mito | |
| | Dehydrogenase | 9 | | subunit | |
| | | | | Alpha | |
| | | 42,211.7 | 8.64 | Subunit | |
| | | 3 | | Beta | |
| 24. | Lipase | 24,903.7 | 6.02 | Red cells | |
| | | 9 | 6.42 | Lyso- | |
| | | 45,415.0 | | somes | |
| | | 2 | | | |
| 25. | Prostate Specific | 84,330.6 | 6.50 | PSA-like | |
| | Antigen (Folate | 9 | | protein | |
| | Hydrolase) | | 6.53 | PSA | |
| | | 50,044.6 | | | |
| | | 1 | | | |
| 26. | Trypsin | 26,558.0 | 6.08 | Cationic | |
| | | 8 | | Trypsin | |
| | | | 4.78 | Anionic | |
| | | 26,487.8 | | Trypsin | |
| | | 0 | | | |
| 27. | Gamma-Glutamyl | 77,257.7 | 5.17 | Tissue | |
| | Transferase | 5 | | | |

It is seen that all of these, without exception, fall in the molecular weight range below 45 kd. Thus is shown that not only do tissues have a small fraction of proteins which are natively in this intermediate to low molecular weight range, but that some of these do leak out during injury.

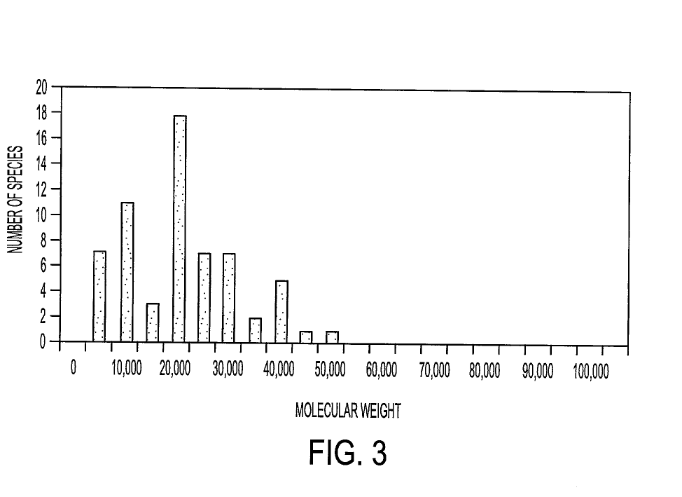

It is important to note that a variety of active factors have been initially discovered in urine. Sixty-two of these are indicated in FIG. 3.

Thus the 2DE pattern shows large number of protein spots that should, in vivo, be rapidly filtered out through the kidney. The answer to this puzzle is simply that nearly all the protein below serum albumin in the 2DE pattern are complexed to form dimmers, trimers, or multimers having such large masses that the kidney retains them. When a gel filtration analysis of serum or plasma is done, it is seen that the expected large peaks representing serum albumin and larger proteins or complexes are seen, but that the absorbance curve drops to the baseline shortly after the albumin has passed, and that almost no absorbing material is seen between albumin and the peaks representing low molecular mass metabolites. Thus the normal kidney retains proteins above approximately 55 kd, and those of lower mass would be expected to be filtered out through the kidney and should appear in the urine.

This expectation is borne out by the results of a gel filtration curve for concentrated urinary proteins. An appreciable fraction of the UV absorbing mass appears after the position where albumin would appear, and before the peaks for low molecular weight metabolites.

These results support experimentally the conventional conclusion that the kidney has a relatively sharp molecular weight cutoff, and that it efficiently removes molecules below approximately 50 kd from the circulation.

2DE analysis of tissues, done under denaturing conditions give a misleading picture since many of the proteins of apparently low molecular mass are actually associated with other proteins or with themselves to give higher mass complexes.

There is little quantitative data on the time course of tissue membrane damage that leads to loss of fluid, salts, metabolites, and then proteins. However edema, followed by malaise and shock are well known. There are no general studies in which the molecular weights of proteins leaking out of cells after injury. Using the methods and systems of the present invention it is feasible to find tissues proteins having a range of native molecular masses, to produce clinical tests for these, and to then relate experimentally the effect of extent of tissue damage and time course of disease on the molecular weights of leakage proteins.

As stated above, the present invention provides for a high-resolution analytical procedure for routine global analysis of proteins found in a bodily fluid, such as urine. A series of automated systems is disclosed for; (a) routinely concentrating proteins from human urine, ranging in size down to approximately 5 kDa, (b) immunosubtracting major proteins from urine to reveal minor proteins, and (c) fractionating protein mixtures on the basis of native molecular weight and isoelectric point applicable to human body fluid proteins.

Such a series of systems now makes it feasible to do large-scale quantitative protein mapping studies. For example, using the instant system, sets of multiple analyses can be run in parallel. In a preferred embodiment, the automated system runs about 200 analyses per day per system. In the context of 2DE, about 100 gels are conducted per day.

By using 2DE to measure the abundance of many proteins, the instant method affords the search for patterns of protein modulation related to disease, as well as for the identification of single protein markers classically used in diagnostics. In one embodiment, such a pattern involving multiple serum proteins is used, but not so limited, to index the human acute phase response in rheumatoid arthritis. Further, in a related aspect, such a pattern can be used to analyze the effects of a drug in tissues.

In another embodiment, a computer means for analyzing 2D gels has been developed to effectively support quantitative studies of large numbers of gels. In one embodiment, the KEPLER™ system (Richardson et al. Carcinogenesis 15(2):325-9 (1994)) has been developed to analyze such large scale studies, and involves an extensive two-dimensional mathematical filter system to remove background, to deconvolute each protein spot into one or more Gaussian peaks, and to calculate the volumes under each peak (representing protein quantity). The position of each peak, and the widths in two dimensions at half height are stored, and a complete pattern of a gel can be very quickly regenerated by such means. All original scan data for each gel is stored, together with the processed data. A multiple montage program allows the comparable areas of a series of up to 1,000 gels to be displayed and inter-compared visually to check on pattern matching. In a related aspect, the KEPLER™ system can place protein abundance data directly in a relational database, allowing the system to cross-reference and inter-compare very large sets (thousands) of gels.

The patterns developed from 2DE can be detected by various means, to include, but not limited to, Coomassie blue and silver staining. In a one embodiment, an ARGENTRON™ automatic silver staining system (see WO 01/16884) is used to increase the sensitivity of detection.

Urinary proteins have been isolated by precipitation with salts or organic acids, by precipitation with a dye, by dialysis, by gel filtration (Anderson et al., Clin Chem (1979) 25:1199-1210; Edwards et al., Clin Chem (1982) 28:160-3; Tracy et al., Appl Theor Electrophoresis (1992) 3:55-65), gel exclusion and centrifugation (Anderson et al., (1979)), by dialysis against high molecular weight compounds (Clark et al., B J Obstet Gynaecol (1984) 91:979-85), by precipitation with acidified acetone (Guevara et al., Electrophoresis (1985) 6:613-19), by ultrafilration (Myrick et al., Appl Theor Electrophoresis (1993) 3:137-146; Gianazzi et al., Electrophoresis (1986) 7:435-438; Gomo et al., Clin Chem (1988) 34:1775-80), or by vacuum dialysis Bueler et al., Electrophoresis (1995) 16:124-34). The key considerations are recovery, loss of low molecular weight constituents, and proteolysis during isolation. In the present invention, many samples must be concentrated reproducibly, thus in one embodiment, gel-filtration and lyophilization techniques are combined to perform the procedure.

The resolving power of current 2DE analyses is essentially limited to a set of the 1,000 to 2,000 most abundant proteins in a sample. With serum, the number resolved is much lower because of the presence of large amounts of albumin, transferrin, haptoglobin, α2-HS glycoprotein, α1-antitrypsin, Gc globulin, α1 acid glycoprotein (orosomucoid), and Ig chains. For urine, similar problems exist due to the presence of IgG, albumin, retinol-binding protein (RBP), transferrin, MAUP (Most Acid Urinary Protein), α1-microglobulin, cystatin C, β2-microglobulin, and Tamm-Horsfall proteins. When these proteins are specifically and quantitatively removed, many new minor proteins are seen. Hence, an aspect of the instant invention focuses on the use of subtraction means to reduce these most abundant proteins, and for concentrating and analyzing the remaining minor ones. By repeating this process cyclically with antibodies against additional sets of proteins, enrichment of low abundance proteins is attained.

In a related embodiment, the Cyclum (Anderson et al., Anal Biochem (1975) 66:159-174 and Anderson et al., Anal Biochem (1975) 68:371-93) system (one of the original recycling affinity chromatographic systems) can be used precisely for the purpose of subtraction of abundant proteins from analyte sample fluids. In a related aspect, the methods such as, but not limited to, frontal subtraction, which is removal of abundant proteins before further analysis, are also useful in this regard. For example, with immunosubtraction as the first fractionation step, other fractionations using different parameters may then be applied.

The spectrum of high resolution multi-dimensional chromatographic methods and the automatic systems for operating them (the PerSeptive Biosystems Integral™ 100Q Multidimensional HPLC System and Pharmacia AKTA system) now allow separation procedures to be precisely defined, and automatically repeated. A wide variety of affinity supports are commercially available which resolve different classes of proteins, for example, enzymes requiring specific cofactors. In a preferred embodiment, size exclusion chromatography (gel filtration) is used as a further dimension to the 2DE system, allowing observations of numerous additional proteins otherwise obscured by high abundance molecules.

Advances in mass spectrometry have now made it possible to determine protein masses up to 20,000 kDa with unit mass accuracy using samples in the picomole or femtomole range. In one embodiment, using Matrix-Assisted Laser Desorption Ionization Time of Flight Mass Spectrometry (MALDI-TOF) and in-source fragmentation, partial sequences of up to 40 amino acids can be obtained (Lennon J J., Protein Sci (1997) 6:2446-53). A variety of methods have been described for recovering proteins from 2D gels for MS analysis (Wilm et al., Nature (1996) 379:466-49). In a related aspect, for the identification of proteins recovered from 2DE gel spots, a PerSeptive Biosystems Voyager DE™ STR BioSpectometer Work Station can be used, which can achieve mass accuracies of <50 ppm and usable sensitivities of 7 femtomole peptide applied to the target. In another related embodiment, for protein fractionation, a PerSeptive Biosystems Integral™ 100Q Multidimensional HPLC System is used. Further, Finnigan LCQ ion trap mass spectrometer and Michrom Magic 2002 microbore HPLC can be used in the systems envisaged for application to the instant invention.

As will be explained in more detail below, the strategy used in the present invention is to first fractionate bodily fluid (e.g., urinary) proteins, analyze each fraction using quantitative high resolution 2DE which resolves 1,000-2,000 proteins per analysis (Anderson and Anderson, Electrophoresis (1978) 17:443-53; Anderson and Anderson, Anal Biochem (1978) 85:331-40; Anderson and Anderson, Anal Biochem (1978) 85:341-54; Anderson et al., Electrophoresis (1995) 16:1977-81; Anderson et al., Toxicologic Pathology (1996) 24:72-76; and Anderson et al., Eli Lily Symposium, 1991, (Probst et al., eds) FASEB, Bethesda, Md. pp. 65-71) and then to seek both qualitative and quantitative changes using an image processing means and analysis programs. Results will be interpreted through reference to the selected databases, to include, but not limited to, Molecular Anatomy and Pathology™ [MAP™], and Molecular Effects of Drugs™ [MED™] databases. Different proteins will be analyzed and identified by mass spectrometry to determine total mass, where fragments masses are produced by proteolysis, and, in addition, the proteins will be partially sequenced by in-source fragmentation (Lennon (1997 supra)) and LC/MS/MS.

Antibodies will be prepared against proteins which are identified as candidate markers, and these used to develop tests for clinical evaluation, and to determine whether the protein antigens are indeed associated with particular disorders (e.g., tumor cells). Antibodies can be made by any conventional method known in the art (such as from polyclonal sera, see, e.g., U.S. Pat. No. 5,480,895). Such methods also include, but are not limited to, the production of monoclonal (see, e.g., U.S. Pat. No. 6,267,959) and phage antibodies (see, e.g., U.S. Pat. No. 6,265,150).

The approach as disclosed in the instant invention is designed to sequentially examine proteins present at successively decreasing abundance levels, with the aim of exhausting available technology in the pursuit of trace proteins. In a related aspect, the goal of the present invention is to identify selective and specific markers for clinical evaluation and use.

Design

In general, the methods increase the number of proteins which can be detected in 2DE patterns of proteins from human body fluids, namely urine. Specifically, methods are disclosed that; (a) concentrate normal and pathological urine proteins, (b) subtract or otherwise fractionate such proteins so that minor proteins can be resolved, (c) allow assembly of a set of test samples, (d) produce annotated references to pathological variation in protein abundance, and (e) improve methods for identifying new proteins by mass spectrometry.

Further, the methods of the instant invention aim to find cancer and other disease, toxicity or drug efficacy indicators searching over a very wide concentration ranges and to apply these methods to a large number of samples. Moreover, the method evaluates candidate differences in marker proteins. In a preferred embodiment, reiterative analyses on whole or fractionated samples are performed to demonstrate the validity of relationships between markers and physiological state. In a related embodiment, by analyzing and evaluating data from large sets of samples, specific tests using such markers are envisaged for clinical application.

In a more preferred embodiment, automation of the preparative and analytical procedures of the design are performed so as to cope with greater number of samples likely to be required to demonstrate statistical significance.

Initial Sample Collection and Processing

Urinary proteins are important as a source of disease marker proteins. However, the dilute nature of urine and the relatively high concentration of a plethora of low molecular weight compounds make it necessary to devote significant effort to the initial preparation of a suitable starting sample of urinary protein. Because of the sometimes large variations in kidney performance between individuals and the consequent large variations in many proteins unrelated to disease, generation of statistically adequate sample sets for marker searches requires the use of automated methods for preparing large numbers of samples.

A normal adult excretes between 20-100 mg of protein/24 hours in a volume of between 600-1600 ml. One average voiding provides ample protein for initial electrophoretic analyses, but not for extended fractionation studies. Individual voiding volumes may be as high as 400 mL. For processing, it is important to decide on maximum sample volume, and to use multiples thereof. In one embodiment, a system has been designed to prepare samples of at least in the amount of 100 mg from individual donors. Previous studies have demonstrated that fresh centrifuged urine can be separated from low molecular weight constituents using large P6 Biogel columns which can be regenerated and used in a cyclic manner (Anderson et al., Anal Biochem (1979) 95:48-61). In a related aspect, to extend the lower mass limits, P2 Biogel can be used. In one embodiment, the products of the first concentration method will be taken up in water and rechromatographed on small P2 gel filtration columns, and the product lyophilized in small bottles which will be sealed, labeled, and stored at −80° C.

Ammonium bicarbonate can be added to the recovered effluent which is then lyophilized, resuspended in water, and re-chromatographed on a small P6 column, relyophilized, and stored at −70° C.

In another embodiment, the system is designed to process one average voiding of approximately 200 mL, and may be expected to yield 10-20 mg of protein from normal urine, and larger quantities from pathological specimens (e.g., >150 mg/d).

In a related embodiment, a 250 mL sample is chosen and the sample vessels will be 200 mL conical centrifuge tubes with screw caps. A support (e.g., Styrofoam box) may be chosen and an internal support provided so that the tubes may be put on crushed ice immediately after collection. Each tube will contain, for example, a tablet of protease inhibitor containing: serine, cystenine and metalloprotease inhibitor.

In accordance with an alternative embodiment of the present invention, to be included in each vessel is a bacteriostatic agent (e.g., 100 mg of sodium azide). Tubes can be centrifuged in refrigerated centrifuges for an appropriate time to pellet desired materials. In a preferred embodiment, each tube may contain a specifically designed insert to keep the pellet at the bottom when the supernatant is siphoned off.

Urinary Protein Preparation

In one embodiment, proteins are prepared by large scale gel filtration and lyophilization. Low temperature gel filtration is used to separate the small amount of protein present from the large amount of low molecular weight materials—chiefly urea and waste metabolic products—present in urine. In another related aspect, column volumes can be investigated by running synthetic urine samples containing a series of low molecular weight compounds. In a related embodiment, evaluation of the systems can be done by adding trace amounts of proteins for which sensitive clinical tests are available (e.g., insulin, IL-6 etc.), and measuring the recovery using commercial clinical laboratory services. In one embodiment (i.e., synthetic urine), proteins are selected for spiking to represent a range of molecular weights and isoelectric points, and preparation methods will be evaluated based on the number and variety of test points recovered with high efficiency. In such an investigation, recoveries are measured and used to set design parameters. Once the design parameters are chosen, the systems are of such flexible design that accommodation of any necessary modifications in volume are readily afforded. Such information will be interfaced with sample data such that supernatants are pumped out of the centrifuge tubes automatically into the column, the completion of this event detected, and each column input line valved over to the elution buffer, (e.g., very dilute ammonium bicarbonate or ammonium formate [i.e., volatile buffers] plus 0.05% sodium azide). Sodium azide combined with precentrifugation should prevent bacterial contamination; however, at intervals, all columns will be repacked and/or sterilized with NaOH. Each protein eluate, as collected, can be frozen. The samples are lyophilized (e.g., a commercial lyophilizer) by a means having the capacity to match the output of the gel filtration system. Overall sample recovery to this point will be determined by diluting and rerunning samples followed by 2DE analysis. If losses due to fly-over occur, the concentration of volatile salts added to the original sample (ammonium formate or bicarbonate) can be increased, and an additional filter added to the lyophilization flasks.

Other systems for cyclically recovering proteins from individual urine samples, and for regenerating and chemically sterilizing the columns between cycles, are envisaged. The system can be monitored by absorbance at 280 nm, cooled, and designed to process at least ten samples per day, more preferably 20 samples a day or still more preferably 100 samples a day.

In another embodiment, the present invention envisages recovery by adsorption to and recovery from a solid phase support, of which C4 and C8 reverse phase media are the preferred candidates. A variety of such supports can be evaluated by exposure to urinary proteins (or synthetic urine), followed by elution in a small volume of suitable solvents, such as 10-50% acetonitrile in aqueous ammonium bicarbonate buffers. In a related aspect, this approach may be combined with prior gel filtration if low molecular weight urine components interfere with protein binding to the supports.

In another embodiment, centrifugal or pressure driven membrane concentrators are employed to retain proteins above 6,000 Da while eliminating most water and low molecular weight substances.

Affinity Matrices

A variety of affinity columns can play a major role in increasing the sensitivity of detection for trace proteins in body fluids such as urine and blood. In one embodiment, reusable columns are preferred because of the lower cost (compared to disposable media) and potentially greater reproducibility. Candidate affinity media is evaluated by use in fractionation of control serum and synthetic or natural urinary protein pools, with bound and unbound fractions analyzed by 2DE to evaluate specificity and capacity. Promising supports are then used in various combinations to achieve the required goal.

For depletion of known high-abundance proteins, immunoaffinity columns using specific monoclonal and polyclonal antibodies are employed. Initial target proteins include but are not limited to, albumin, tranferrin, α1antitrypsin and α2macroglobulin. A secondary list includes, but is not limited to, α1acid glycoprotein, C3, hemopexin, α2HS glycoprotein, α1antichymotrypsin, Gc globulin and ceruloplasmin. In each case, antibody preparations (whole antiserum, Ig fraction of antiserum, monoclonal ascites or tissue culture supernatant) are subjected to affinity purification on columns of purified antigen (commercially available isolated human serum protein) to ensure specificity. These isolated specific antibodies can then be covalently coupled to suitable solid phase supports. Methods for attaching such antibodies to solid phases can be by any means known in the art (see, e.g., U.S. Pat. Nos. 5,773,308 and 5,861,319). Supports will be selected for stability and high flow rate. In one embodiment, the use of POROS perfusion chromatography supports is preferred.

For some proteins, effective non-immunological affinity matrices exist. For example, human serum immunoglobulins (particularly IgG, IgA and IgM) bind effectively to proteins A and G from bacterial sources, and covalently bound suitable supports comprising these proteins are available commercially. In a related aspect, haptoglobin may be removed using a column of immobilized human hemoglobin.

Taken together, these specific affinity supports are capable of removing approximately 95% of the total protein in serum and urine. The unbound fraction can then be analyzed at approximately 20-fold higher 2DE loading than whole urine. The bound and eluted fractions, when pooled, can be similarly analyzed to quantify major protein abundance.

In another aspect, group-specific supports such as lectins can also be used. By employing lectins specific for various sugar structures, serum and urinary glycoprotein fractions can be obtained as an enriched fraction for identification and isolation of select markers.

Additional, less specific affinity media can also be used to enrich fractions for potentially useful markers. These supports include, but are not limited to the following: arginine and benzamidine, glutathione, Cibachron Blue, calmodulin, gelatin, heparin, lysine, Procion Red HE-3B, nucleic acids and metal affinity columns (serum proteins, Porath and Olin, Biochemistry (1983) 22:1621).

Additional applications of immunosubtraction techniques related to protein subtraction and assay will be explored below. When tens or hundreds of similar gels are being analyzed, corresponding spots may be recovered from a large number of gels, pooled and the proteins isolated by published electrophoresis methods and proprietary modifications of them (see, e.g., U.S. Pat. No. 4,824,547). The small amount of antigen may be used to prepare both a small column and to produce antibodies. In a hundred or so cycles, specific antibodies may be prepared, which in turn is used to prepare an antibody column that serves to subtract and to purify, in a cyclic mode, more antigen. In one embodiment, when a specific protein (antigen) of interest becomes apparent, the first step is to determine whether any of the multivalent sera have useful quantities of antibody of information content. In a related aspect, additional purification steps may be required to purify both the final antigen and antibody products.

In accordance with the present invention, the basic techniques in preparing broad-range immunosubtractive columns are to prepare one starting antiserum, isolate specific IgG using a column of immobilized antigen mixture, and prepare a column which will subtract part of the starting antigen population. The unbound antigen is then used to produce a new antiserum and the steps are repeated. The advantage of this system is that once a reasonably balanced column (or serially arranged set of columns) is produced, a variety of samples may be eluted comprising various combinations of components. In a related aspect, 2DE is used to evaluate performance of the column.

Fractionation Based on Mass and IEF of Native Proteins

Fractionation based on native protein mass, followed by 2DE under denaturing conditions allows many protein subunits to be associated with their native configuration. This is especially true with very large protein such as lipoproteins which yield very small subunits with SDS.

For highest resolution, urine is resolved (cytosols for example) into fractions by gel filtration then recovered for analysis under denaturing conditions by 2DE. Quantitative analysis allows for identification of subunits of specific complexes, and the stoichiometry of each in the native molecule. The importance of this technology is that it allows not only a reduction in the complexity of mixture (by separating more rare proteins from the more abundant ones) and the concentration of trace components, but more precise characterization of new proteins in terms of their molecular associations. The key component in this regard is the characterization of a range of gel filtration media to select optimal resolution of high and low abundance urinary proteins, and to attain the highest practical flow rate (a critical determinant of sample throughout, since most resolution gel filtration media require very low flow rates and runtimes of 6-12 hours per sample).

In addition, the present invention allows for analysis of a range of other methods for fractionation of native urinary proteins. Prominent among these (but not limited to) are native isoelectric focusing (IEF) and centrifugation. In a preferred embodiment, IEF is conducted with flatbed IEF and/or by column chromatofocusing. In a related aspect, present flat-bed electrophoresis systems are not preparative. In another embodiment, cooled flat-bed systems having volumes of several hundred milliliters. In a related aspect, a flat bed system is constructed with beryllium oxide plate cooling.

In one embodiment, for native protein isoelectric focusing agar, urine-fractionate and ampholytes have been mixed together warm, and allowed to set. Cut out bands are then allowed to focus in the long dimension giving a very shallow pH gradient, and allowing all protein of a very narrow isoelectric point range to be focused and recovered. Chromatofocusing can be carried out using commercially available systems and centrifugation can be performed using zonal sedimentation on density gradients.

Modification of Systems to Encompass Low Molecular Weight Proteins and Peptides

Current 2DE analyses cut off at approximately 6,000 kDa or slightly higher. Since known active peptides in urine extend to a lower mass range, it is essential to be able to extend downward the molecular weight range covered. One dimensional SDS systems have been developed which extend the range resolved down to approximately 2.5 kDa using 18% Tris-Glycine gels, and even lower with tricine or MES gels. Thus, systematic analysis of different gel concentrations and different buffers are used to extend the range of molecular weights detected in the ISO-DALT® 2DE system to as low as possible.

In accordance with the present invention, to achieve detection of low molecular weight product, modulation of buffer composition is first carried out followed by changes in the concentration of the acrylamide gels; specifically increasing the gel concentration at the lower end of the gradient gel. The present invention also envisages the preparation of slab gels using a proprietary computer-controlled large volume gradient delivery system which allows systematic variation in gel % T gradient (see, e.g., U.S. Pat. Nos. 6,245,206; 6,136,173; 6,123,821; and 5,993,627). In another embodiment, a series of changes in the ratio between acrylamide and bisacrylamide are performed to manipulate pore size. Physical chemical studies on acrylamide gels have shown that increasing the amount of cross linker (bisacrylamide) produces smaller pore sizes, and hence the resolution of small molecules. Another embodiment includes addition of linear acrylamide in the gel to partially obstruct pores, and effectively lower pore size.

In general, low molecular weight peptides tend to diffuse out of gels during washing, fixing and staining faster than do larger ones. This appears to be especially true when the proteins are covered with SDS. Additionally, low molecular weight protein spots diffuse more during electrophoresis, and hence give larger spots. To circumvent these problems, 2DE is modified such that the gels are run faster (i.e., in 5 hr instead of the typical 18 hr overnight run). Further, new cooling methods and means are disclosed below to allow for the increased running time without consequential loss in resolution (e.g., “smiling” effects). In another embodiment, fixing and staining procedures have been modified to immobilize the small peptides faster. In a related aspect, this can be accomplished by increasing the alcohol concentration during initial fixation, and by inclusion of glutaraldehyde during the fixation process. In another embodiment, Coomassie Blue has been used as a potent protein fixative (e.g., stained gels show negligible loss of protein over months when stored in water). Hence the inclusion of Coomassie Blue in initial washing is also available as a means to reduce protein loss using the present invention.

Development of Routine Mass Spectrometric Analysis of Proteins from Gels

Mass spectrometric analyses are now an essential aspect of 2DE studies, providing a beautiful and elegant solution to the problem of identifying very small protein samples. A variety of methods have been developed for analyzing proteins from gels by mass spectrometry (Wilm et al. (1996); Jungblut and Thiede, Mass Spectrom Rev (1997) 16: 145-62; and Li et al., Electrophoresis (1997) 18:391-402). In accordance with the present invention, an automatic scanner allows spots to be located on wet gels, identified by position, and cut out using a small robotic punch which expels each protein into a separate well on a 96 well microtiter plate.

In a related aspect, the instant invention provides for automatically recovering sufficient protein from each spot, optionally digesting it with a proteolytic enzyme, and then spotting each on an MS target plate.

As the number of 2DE analyses and MS analyses increases, means are required for integrating the two so that the investigator examining a large set of gels (for example sets which resolve urinary proteins from different groups of cancer patients) can not only examine and inter-compare gel patterns but can also review MS data for individual spots. This requires both a new level of automation in the acquisition of MS data, and development of new programs to integrate the two information sources together. Not all protein on all gels can be analyzed. Hence analyses fall into two groups, namely those done for master gel patterns, and those done for identity confirmation when a protein is found to vary in an interesting manner, or to identify a new protein.

According to the present invention, two general methodologies are used for MS analysis on 2D gels. In one embodiment, the protein pattern is transferred to a porous membrane, usually composed of nitrocellulose. Then these may be stored, and individual spots cut out and analyzed or sections of the membrane may be inserted into the MALDI TOF mass spectrometer and scanned. In either instance, matrix must be applied to the membrane. In a modification of this approach, the cut out spots are dissolved in a suitable solvent, matrix added, and the solution applied to the target and dried. A major point in this approach is that the unused portions of the membrane may be stored.

In another embodiment, gel spots are cut out and processed to remove protein that may be analyzed directly, or after enzyme digestion. This may be done in microtiter plates.

Integration of Components for Automated High-throughput Serum and Urine Sample Analysis

According to the present invention, 2DE technology is used to analyze fractionated test samples generated during the processes as disclosed above. The quantitative protein abundance data obtained by 2DE is then combined with clinical information to select candidate marker proteins (CMPs). By using 2DE to measure the abundance of many proteins rather than a few, a means is provided to search for patterns of protein abundance changes related to disease, as well as for the single protein markers classically used in diagnostics.

In a preferred embodiment, protein samples can be prepared by solubilization of aliquots in a six-fold excess of (V/V) of 9M urea, 2% NP-40 detergent, 0.5% dithiothreitol, 2% pH 8.0-10.5 Pharmalytes. The resulting solubilized protein samples can be stored at −80° C. as aliquots in labeled vials.

In another preferred embodiment, sample proteins can be resolved by 2-D electrophoresis using the 20×25 cm ISO-DALT® 2-D gel system operating with 20 gels per batch. In a related aspect, all first dimension isoelectric focusing gels can be prepared using the same single standardization batch or ampholytes (BDH 4-8A). The gels can be run for 34,500 volt-hours using a progressively increasing voltage protocol implemented by a programmable high voltage power supply.

In one embodiment, an Angelique™ computer-controlled gradient casting system will be used to prepare second dimension SDS gradient slab gels in which the top 5% of the gel is 11% T acrylamide, and the lower 95% of the gel varies linearly from 11% to 18% T. Each gel can be identified by a computer-printed filter paper label polymerized into the gel. In a related aspect, first dimension IEF tube gels will be loaded directly onto the slab gels without equilibration, and held in place by agarose. In a further related aspect, second dimension slab gels are run in groups of 20 in thermostable DALT tanks with buffer circulation.

According to the present invention, gels can be stained by a colloidal Coomassie Blue G-250 procedure in covered plastic boxes. This procedure involves fixation of sets of gels in a buffer comprising ethanol and phosphoric acid. Further, the procedure includes three washes in cold ionized water, transfer to a methanol, ammonium sulfate, phosphoric acid buffer, followed by addition of a gram of powdered Coomassie Blue G-250 stain. Staining requires approximately 4 days to reach equilibrium intensity. Gels are subsequently be silver-stained using an Argentron™ automated silver stain system.

All run parameters, reagent sources and a lot of information, and notations of deviation from expected results are recorded in a computerized database specially designed for 2DE applications.

Each stained slab gel is digitized using a CCD scanner. Each 2D gel is processed using the LSB Kepler® software system to yield a spot-list giving position, shape and density information for each detected spot. Processing parameters and file locations are stored in a relational database, while various log filed detailing operation of the automatic analysis software are archived with the reduced data. The computed resolution and level of Gaussian convergence of each gel is inspected and archived for quality control purposes. The image processing methodology used for silver-stained images is based on a similar protocol optimized for the higher density images produced by the silver stain.

In matching individual gels to the chosen master 2-D pattern, a series of about 50 proteins is matched with a montage of all the 2-D patterns in the experiment. Subsequently, an automatic program is used to match additional spots to the master pattern using as a basis, the manual landmark data entered by an operator. After the automatic matching (when 500-900 spots have been matched on each gel), an operator inspects matching for spots considered important to the experiment.

The groups of gels making up the experiment are scaled together (to eliminate quantitative difference due to gel loading or staining differences) by a linear procedure based on a selected set of spots. These spots are selected by a procedure which selects spots which have a good initial intra-group CV, have a good (non-elongated) shape, an integrated density between certain limits (avoiding very small or overloaded spots) and are detected on almost all gels of the set. All gels in the experiment are scaled together by setting the summed abundance of the selected spots equal to a constant (linear scaling).

Statistically significant differences will initially be defined as proteins showing t-test values of P<0.001 effects when experimental protein abundance (integrated spot optical densities after Coomassie Blue staining or silver staining) is compared against appropriate disease group of samples against controls. Candidate marker proteins will be selected through statistical comparison of an appropriate disease group of samples against controls, followed by comparison against the results of other cancers to assess specificity. In a preferred embodiment, interesting candidates will be further evaluated by correlation of CMP abundance with clinical data associated with severity or duration of disease. In a related aspect, the development of more sophisticated statistical approaches will be assessed and acquired as the project proceeds.

Development of Provisional Assays for Candidate Marker Proteins

The strategy of the present invention is based on 2DE analyses which yield sufficient physical mass of protein for mass spectrometric identification and for antibody production, if necessary. This strategy requires that a sufficient number of 2DE-based assays be performed to conclude that a candidate marker has, indeed, been found. The next step requires characterization by mass spectrometry. While such assays do not deliver the information return of 2DE (yielding, as they do, data on only one protein), they can be much cheaper and faster, and are thus applicable to the large sets of samples required to validate the specificity and sensitivity to a CMP

In one embodiment, a PerSeptive Biosystems Integral 100Q workstation together with ID sensor cartridges to which are bound antibody specific for a CMP is used. In a related aspect, cartridges will be made using antibodies generated in rabbits and protein excised from analytical 2DE gels run during the processes referred to above. In another embodiment, sufficient sequence information is generated to allow peptides to be synthesized and used for antibody production, or such data will be used to produce a probe which will allow the gene for the candidate marker to be cloned and expressed. In another related aspect, Integral/ID sensor configuration allows a simple capture/elution cycle to be run in <4 min, with sensitivities for eluted analyte of 100 ng to 10 μg in any applied sample volume ranging from 5 μl to 1 ml using UV detection at 280 nm. In a separate related aspect, an enzyme-conjugated second antibody can be added to the system, and a cleavable substrate added suitable for detection sensitivities of 125 pg and 2 pg, respectively. The strengths of this assay system are the ability to rapidly prototype the assay (given an antibody), and then to use it to assay 100-1,000 samples in a period of one to five days. In the event that more widespread testing is required using lower cost equipment, implementation of a 96-well plate format ELISA or other suitable assay will be performed.

The following non-limiting examples illustrate the efficacy and advantages associated with the analysis system for certain body fluids (i.e., urine) in accordance with the present invention. It is understood that these examples are for illustration purposes only and that alternative embodiments, such as the use of similar size exclusion gels and alternative chromatographic and hydrodynamic techniques, are contemplated as within the scope of the present invention.

### EXAMPLES

Materials

2D Equipment

An ISO-DALT® 2-D gel electrophoresis system (Large Scale Biology Corp. [LSBC] Germantown, Md.) for automated two-dimensional electrophoresis (2DE) analyses currently supporting throughput of 200 gels per day per module are used and is partially described in U.S. Pat. No. 5,993,627. The 2-D equipment includes: six 20-place ISO units for casting and running first dimension gels; six 20-place casting boxes for 8″×10″ format slabs; three 40-place casting boxes for 5″×7″ format slab gels; one 10-place and four 20-place DALT tanks for running second dimension slab gels: an Angelique™ computer-controlled gradient maker for reproducibly casting polyacrylamide gradient gels to user-defined or preset specifications; a thermostatic cooling system for the DALT tanks; flat bed and advanced vertical (IsomorpH™) isoelectric focusing apparatus; blotting apparatus especially designed for large format ISO-DALT gels; power supplies; large capacity shaker; slab gel cassette washing machines; and large light box. Scanners include Eikonix 1412 (4K×4K), Princeton Instruments (1K×!K cooled CCD) and Apogee Instruments (1.5K×1K CCD) devices for absorbance and fluorescence gel scanning.

For the identification of proteins recovered form 2DE gel spots, a PerSeptive Biosystems Voyager DE™ STR BioSpectrometer Work Station is used. For fractionation, a PerSeptive Biosystems Biovision 100Q Multidimensional HPLC System is used. A Finnigan LCQ ion trap mass spectrometer and Michrom Magic 2002 microbore HPLC also are employed.

Data from protein separations are extracted, analyzed and organized using the Kepler 2-D and 1-D gel analysis software systems and the VKPL software, a modified version of the Kepler® software (WO01/26039) and the Oracle Rdb relational database system with SQL interface. Software development tools include Fortran and C compliers; X-windows, Motif, Windows NT and Web graphical interface development software; and SAS statistical software.

Methods

Collection of Urine Specimens

Random urine specimens (approximately 200 ml each) were collected from normal individuals who did not have sign of any disease or illness at the time of collection of urine samples. The specimens were collected in sample tubes in which the following buffer and protease inhibitor mixtures were previously added: a) Two tablets of mini protease inhibitor (Sigma), b) 290 mg of Sigma phosphate buffer.

Immediately after collection of samples, the contents of the tubes were mixed well to dissolve the buffer and inhibitor tablet. Urine samples collected in this method contains various components such as red cells, white cells, casts etc., which interfere with the downstream processes that are necessary to concentrate urinary proteins. These “unwanted” cells and casts were removed by centrifuging the urine samples (within half an hour of collection) for 20 min at 2500 rpm. The supernatant containing urine proteins were then transferred to a centrifugal filter device, and centrifuged at 3200 rpm until the entire sample volume was filtered out.

Exchange of Buffer in Urine Proteins:

Because urine samples contain large quantities of small molecular weight salts and metabolic by-products, it is important to exchange buffer in concentrated urine proteins. In the present method, 7-8 ml of buffer A (100 mM Na₂HPO₄, 150 mM NaCl, 0.02% NaN₃, and one mini protease inhibitor tablet (Sigma) per 10 ml of buffer) were added to the filter device to dilute the concentrated protein solution. Using a dropper, the concentrated protein solutions were resuspended thoroughly with the buffer already added to it. The resuspended solution was then centrifuged further at 3200 rpm until the buffer concentrated down to less than a volume of 1 ml. This step removes some fractions of small molecules (such as urea, uric acid etc.) present in urine. The concentrated samples were collected by inverting the filter device, and by centrifugation at 2000 rpm for 1 min. The sample volume at this stage is in the range of 0.5 to 1 ml.

Fractionation of Urine Proteins on the Basis of Their Native Molecular Weight

Fractionation of urine proteins was done by using a Superdex 75 gel filtration column. Superdex 75 was chosen as the matrix of interest because the size fractionation range for this matrix is 3-75 kDa. Two fractions were generated at > and <30 kDa. The proteins in the >30 kDa fraction were considered as the high molecular weight fraction, and the <30 kDa fraction was considered as the low molecular weight fraction. These fractions were concentrated using a centrifugal filter device with a 5 kDa molecular weight cut off.

Immunosubtraction of Urinary Proteins in the High Molecular Weight Fraction:

The high molecular weight fraction contains a large quantity of abundant proteins such as albumin and α1-acid glycoproteins. To get high resolution 2D gel pattern, it was important to specifically remove these abundant proteins from the high molecular weight fraction. Therefore, an immunoaffinity column containing immobilized antibodies for albumin and α1-acid glycoprotein was prepared. Briefly, polyclonal antibodies to each were separately immobilized in separate columns and individual binding capacity of each column was determined. The solid phase material from each column was combined to give a binding capacity proportional to the normal concentrations of albumin and α1-acid glycoproteins in urine. The samples were loaded in the immuoaffinity column, and the eluted volumes were collected and concentrated using centrifugal filter devices.

To prepare urine samples ready for 2D electrophoresis, it is also important to exchange the buffer of concentrated solution with volatile ammonium bicarbonate buffer (Werner et al., Clin Chem (1993) 39:2386-96). The buffer solution used for this purpose contained one mini protease inhibitor tablet (Sigma) per 10 ml volume. The exchange of buffer with ammonium bicarbonate involved several steps. In the first step, approx. 1 ml concentrated protein sample was taken in a small size filter device. The sample was diluted in the filter device with 4.5 ml with NH₄HCO₃ buffer, and centrifuged at 32,000 rpm until the volume decreased to 0.5 ml. This step was repeated twice. The final volume of the sample was around 0.5 ml. Finally, the concentrated samples were lyophilized over a period of 18 hrs and dissolved in an appropriate volume of CHAPS containing protein solubilizing solution.

2DE of Urinary Proteins

Protein samples are prepared by solubilization of aliquots in a six-fold excess of (V/V) of 9 M urea, 2% non-ionic detergent, 0.5% dithiothreitol, 2% pH 8.0-10.5 Ampholytes. The resulting solubilized protein samples will be stored at −80° C. as aliquots in labeled vials.

Sample proteins were resolved by 2-D electrophoresis using the 20×25 cm ISO-DALT 2-D gel system. All first dimension isoelectric focusing gels are prepared using the same single standardized batch of ampholytes selected by a batch testing program for database work. Ten microliters of solubilized protein are typically applied to each gel, and the gels run for 34,500 volt-hours using a progressively increasing voltage protocol implemented by a programmable high voltage power supply.

An Angelique™ computer-controlled gradient casting system (LSBC) is used to prepare second dimension SDS gradient gels in which the top 5% of the gel is 11%T acrylamide, and the lower 95% of the gel varied linearly from 11% to 18%T. Each gel is identified by a computer-printed filter paper polymerized into the gel. First dimension IEF tube gels are loaded directly onto the slab gels without equilibration. Second dimension slab gels are run in groups of 20 in thermostable DALT tanks with buffer circulation.

Gels will be stained by a colloidal Coomassie Blue G-250 procedure in covered plastic boxes, with 10 gels per box. This procedure involves fixation of sets of ten gels in 1.5 liters of 50% ethanol/2% phosphoric acid overnight, three 30-minute washes in 2 liters of cold deionized water, and transfer to 1.5 liters of 34% methanol/17% ammonium sulfate/2% phosphoric acid for one hour followed by addition of a gram of powdered Coomassie Blue G-250 stain. Staining requires approximately 4 days to reach equilibrium intensity. Gels are subsequently be silver-stained using the Argentron™ Silver staining.

The image processing methodology used for gel images involved digitizing each gel in red light at 133 micron resolution, using an Eikonix 1412 scanner. Each 2-D gel is processed using KEPLER™ software system to yield a spotlist giving position, shape and density information for each detected spot. This procedure makes use of digital filtering, mathematical morphology techniques and digital masking to remove background, and uses full two-dimensional least-squares optimization to refine the parameters of database, while various log files detailing operation of the automatic analysis software are archived with the reduced data. Silver-stained images are processed by a similar procedure optimized for the denser images produced by silver.

In matching individual gels to the chosen master 2-D pattern, a series of about 50 proteins is matched by an experienced operator working with a montage of all the 2-D patterns in the experiment. Subsequently, an automatic program is to be used to match additional spots to the master pattern using as a basis, the manual landmark data entered by the operator. After the automatic matching (when 500-900 spots have been matched on each gel), the operator inspects matching for spots considered to the experiment.

The groups of gels making up the experiment are scaled together (to eliminate quantitative differences due to gel loading or staining differences) by a linear procedure based on a selected set of spots. These spots are selected by a procedure which selects spots which have a good initial intra-group CV, have a good (non-elongated) shape, an integrated density between certain limits (avoiding very small or overloaded spots) and are detected on almost all gels of the set. All gels in the experiment are scaled together by setting the summed abundance of the selected spots equal to a constant (linear scaling).

Statistically significant differences are defined as proteins showing a t-test value of P<0.001 when experimental protein abundance (integrated spot optical densities after Coomassie Blue staining or silver staining) is compared against appropriate controls.

Spots were cut from gels, digested with trypsin (generally based on the in-gel tryptic digestion method of Rosenfeld et al., [Anal Biochem (1992) 203:173-79] with modifications) and analyzed by MALDI-TOF-MS and LC/MS/MS.

Mass Spectrometric Detection of Proteins

Cut spots are placed in separate wells on a solid phase surface. Samples are digested in situ with trypsin as follows: 3 μl of trypsin (30 ng/μl) and the samples were incubated at room temperature for 5 min. A sufficient volume of 0.2M NH₄HCO₃ is added to ensure complete submersion of the cut gel spots in the digestion buffer. Samples were incubated overnight at 37° C. All samples are acidified with 1 μl glacial acetic acid. The samples are dried and reconstituted in 1% glacial acetic acid for subsequent mass spectral analysis.

Trypsinized proteins were further prepared using α-cyano-4-hydroxycinnamic acid as the MALDI matrix. The matrix solution was saturated in 40% CH₃CN, 0.1% trifluoroacetic acid (TFA) in water. The spots are applied first to the smooth, solid phase, then 20 μl of matrix solution is added in with a pipette tip and the sample allowed to air evaporate.

MALDI experiments were performed on a Bruker Biflex time-of-flight mass spectrometer equipped with delayed ion extraction. A pulsed nitrogen laser was used for all of the data acquisition. The performance of the mass spectrometer produced sufficient mass resolution to produce the isotopic multiplet for each ion species below mass-to-charge (m/z) ratio of 3000. The data was analyzed using existing software.

All MALDI mass spectra were internally calibrated using masses from two trypsin autolysis products (monoisotopic masses 841.50 and 2210.10). Mass spectral peaks were determined based on a signal-to-noise (S/N) ratio of 3. Two software packages, Protein Prospector and Profound, were used to identify protein spots. The human, rat and mouse nonredundant (nr) database consisting of SwissProt, PIR, GeneBank and OWL were used in the searches. Parameters used in the searches included proteins less than 100 kDa, greater than 4 matching peptides and mass errors less than 45 ppm.

Automated analysis of peptide tandem mass spectra was performed using the SEQUEST computer algorithm (Finnigan MAT, San Jose, Calif. ). The non-redundant (NR) protein database was obtained as an ASCII text file in FASTA format from the National Center for Biotechnology Information (NCBI).

Anderson, N. G. Analytical techniques for cell fractions. VIII. Analytical differential centrifugation in angle-head rotors. Analytical Biochemistry 23: 72-83, 1968.

Tietz Textbook of Clinical Chemistry. Burtis, C. S., ed. W. B. Saunders Co., 3rd ed., 1998.

Silver, I. A.,Local PO2 in relation to intracellular pH, cell membrane potential and potassium leakage in hypoxia and shock. In: Adv Exp Med Biol (1973) 37A:223

Nungester W J, Adair J A, Allardyce R A, Paradise L J Titration of cytolytic antitumor antibody utilizing absorbance at 260 nm to measure the leakage of cell constituents. In: Cancer Res (June 1969) 29(6):1262-6

Watanabe S, Hioki M, Mohri T, Kitagawa H Transaminases of hepatic tissue culture cells and the effect of carbon tetrachloride on their leakage. In: Chem Pharm Bull (Tokyo)(May 1977) 25(5):1089-93

Hansen K N, Bjerre-Knudsen J, Brodthagen U, Jordal R, Paulev P E Muscle cell leakage due to long distance training. In: Eur J Appl Physiol (1982) 48(2):177-88

Roxin L E, Hedin G, Venge P Muscle cell leakage of myoglobin after long-term exercise and relation to the individual performances. In: Int J Sports Med (October 1986) 7(5):259-63

Yamamoto H, Kuchii M, Masuda Y, Murano Studies on the function of cell membrane. 1. Leakage of NADH-cytochrome c reductase into plasma from liver cells in CC4-poisoned rats. In: Jpn J Pharmacol (April 1973) 23(2):141-50

Takami H, Matsuda H, Kuki S, Nishimura M, Kawashima Y, Watari H, Furuya E, Tagawa K. Leakage of cytoplasmic enzymes from rat heart by the stress of cardiac beating after increase in cell membrane fragility by anoxia. In: Pflugers Arch (April 1990) 416(1-2):144-50

Sullivan K A, Berke G, Amos B 51 Cr leakage from and uptake of trypan blue by target cells undergoing cell-mediated destruction. In: Transplantation (June 1972) 13(6):627

Nilsson K, Ekstrand B. The effect of storage on ice and various freezing treatments on enzyme leakage in muscle tissue of rainbow trout (Oncorhynchus mykiss). In: Z Lebensm Unters Forsch (July 1993) 197(1):3-7

Boston P, Jackson P. Purification and properties of a brain-specific protein, human 14-3-3 protein. In: Biochem Soc Trans (October 1980) 8(5):617-8

Jackson P, Thompson R J High-resolution two-dimensional analysis of human brain soluble proteins. In: Biochem Soc Trans (October 1980) 8(5):616-7

Jackson P, Thomson V M, Thompson R J Demonstration of basic human-brain-specific proteins by the BASO-DALT system. In: Clin Chem (April 1982) 28(4 Pt 2):920-4.

Boston P F, Jackson P, Kynoch P A, Thompson R J Purification, properties, and immunohistochemical localisation of human brain 14-3-3 protein. In: J Neurochem (May 1982) 38(5):1466-74

Jackson P, Thompson R J The demonstration of new human brain-specific proteins by high-resolution two-dimensional polyacrylamide gel electrophoresis. In: J Neurol Sci (March 1981) 49(3):429-38

Klose J, von Wallenberg-Pachaly H Changes of soluble protein populations during organogenesis of mouse embryos as revealed by protein mapping. In: Dev Biol (Jul. 15, 1976) 51(2):324-31

Jungblut P, Zimny-Arndt U, Klose J Composition and genetic variability of proteins from nuclear fractions of mouse (DBA/2J and C57BL/6J) liver and brain. In: Electrophoresis (July 1989) 10(7):464-72

Gauss C, Kalkum M, Lowe M, Lehrach H, Klose J Analysis of the mouse proteome. (I) Brain proteins: separation by two-dimensional electrophoresis and identification by mass spectrometry and genetic variation. In: Electrophoresis (March 1999) 20(3):575-600

Jungblut P, Baumeister H, Klose J Classification of mouse liver proteins by immobilized metal affinity chromatography and two-dimensional electrophoresis. n: Electrophoresis (July 1993) 14(7):638-43

Jungblut P, Klose J Dye ligand chromatography and two-dimensional electrophoresis of complex protein extracts from mouse tissue. In: J Chromatogr (Nov. 17, 1989) 482(1):125-32

Klose J Fractionated extraction of total tissue proteins from mouse and human for 2-D electrophoresis. In: Methods Mol Biol (1999) 112:67-85

Klose J Large-gel 2-D electrophoresis. In: Methods Mol Biol (1999) 112:147-72

Jungblut, P., and Klose, J., Composition and genetic variability of heparin-sepharose CL-6B protein fractions obtained from the solubilized proteins of mouse organs. In: Biochem Genet (December 1986) 24(11-12):925-39

Anderson, N. G., Anderson, N. L., Tollaksen, S. L., Hahn, S. L., Giere, F., and Edwards, J. Analytical techniques for cell fractions. XXV. Concentration and two dimensional electrophoretic analysis of human urinary proteins. Anal. Biochem. 95: 48-61, 1979.

Tollaksen, S. L., and Anderson, N. G., Two dimensional electrophoresis of human urinary proteins in health and disease. Electrophoresis '79. B. Radola, ed., W. de Gruyter, Berlin, pp 405-414, 1980.

Edwards, J. J., Anderson, N. G., Tollaksen, S. L., et al. Proteins of human urine II. Identification by two-dimensional electrophoresis of a new candidate marker for prostate cancer. Clin. Chem. 28: 160-163, 1982.

Grover P. K., Resnick, M. I. High resolution two-dimensional analysis of urinary proteins of patients with prostatic cancer. Electrophoresis 18: 814-818, 1987

Marshall, R. Anal. Chem. 340: 340-346, 1984.

Clark, P. M. S., Power, G. M., Whitehead, T. P., Electrophoresis '82, pp 435-444, 1983.

Marshall, T. Williams, K. M., and Vesterberg, O. Electrophoresis 6: 47-52, 1985.

Myrick, J. E., Caudill, S. P., Robinson, M. K., Hubert, I. L., Appl. Theoret. Electrophoresis 3: 137-146, 1993.

Rasmussen, H. H., Orntoft, T. F., Wolf, H., and Celis, J. E. Towards a comprehensive database of proteins from the urine of patients with bladder cancer. U. Urol. 155(6), 2113-9, 1996.

Gianazzi, A., Atrua-Testori, S., Rihetti, P. G., Bianchi-Bosisio, A., Electrophoresis 7:435-438, 1993.

Tracy, R P., Young, D. S., et al., Appl. Theoret. Electrophoresis 3: 55-65, 1992.

Bueler, M. R., Wiederkehr, F., Vondershnitt, D. J., Electrophoresis 16: 124-134, 1995.

It will be understood that various modifications may be made to the embodiments disclosed herein. Therefore, the above description should not be construed as limiting, but merely as exemplifications of preferred embodiments. Those skilled in the art will envision other modifications within the scope and spirit of the claims appended hereto.

All patents and references cited herein are explicitly incorporated by reference in their entirety.

1. A method of detecting at least one low molecular weight protein and/or peptide component in a biological fluid comprising:
a) fractionating proteins or peptides by molecular weight, separating fraction having a molecular weight above about 3 kDa and below the filtration limits of a normal kidney; and
recovering each fraction and determining the proteins or peptides present.

2. The method of claim 1, wherein the results of said detection are used to generate an cognizable pattern of low molecular protein and/or peptide components comprising said biological sample which can be correlated with physiological state.

3. The method of claim 1, wherein said biological fluid is selected from the group consisting of urine, blood, tissue cytosol or other fluid, cerebral spinal fluid, sputum, feces and sweat.

4. The method of claim 1, wherein said biological fluid is urine.

5. The method of claim 1, wherein said concentrating step comprises separation of low molecular weight constituents by size exclusion chromatography.

6. The method of claim 5, wherein said separation comprises sequential chromatography by separate stationary phases comprising different mesh sizes.

7. The method of claim 1, wherein said concentrating step comprises addition of at least one protease inhibitor to the body fluid upon collection.

8. The method of claim 1, wherein said concentration step comprises a hydrodynamic step.

9. The method of claim 8, wherein said hydrodynamic step is centrifugation.

10. The method of claim 1, wherein said fractionating step comprises the elution from a reverse phase stationary phase.

11. The method of claim 10, wherein said reverse phase is a non-porous C18 material.

12. The method of claim 1, wherein said fractionating step further comprises elution from an affinity column.

13. The method of claim 12, wherein said affinity column comprises monoclonal polyclonal or recombinant microorganism display antibodies.

14. The method of claim 14, wherein said monoclonal and/or polyclonal antibodies are directed to target proteins selected from the group consisting of albumin, tranferrin, α₁antitrypsin, and α₂macroglobulin, α₁acid glycoprotein, C3, Tamm-Horsfall protein, hemopexin, α₂HS glycoprotein, α₁antichymotrypsin, Gc globulin and ceruloplasmin.

15. The method of claim 13, wherein said affinity chromatography is a non-immunologic entity comprising matrix.

16. The method of claim 16, wherein said non-immunologic entity is selected from the group consisting of protein A, protein G, haptoglobin, arginine, benzamidine, glutathione, Cibachron blue, calmodulin, gelatin, heparin, lysine, lectins, Procion Red HE-3B, nucleic acids and metal affinity media.

17. The method of claim 1, wherein said separating step comprises two-dimensional electrophoresis (2DE).

18. The method of claim 1, wherein said separating step comprises zonal sedimentation centrifugation on density gradients.

19. The method of claim 1, wherein said deflecting step comprises time of flight mass spectrometry or liquid chromatography.

20. An image comprising a pattern of data generated from step (c) of claim 1, wherein said image provides linkage to an annotation comprising data selected from the group consisting of patient data, sequence data, antibody selection, physicochemical protein data, protein abundance data and synthesis correlation data between modulation of said protein abundance and physiological state.

21. The image of claim 20, wherein said image is formed through an image data storing means, said image data being produced from an image of a stationary phase and stored on said storage means, further wherein said image is displayed on a displaying means based on an the image data stored in the image data storing means, wherein said image displaying means is adapted to display the image and pattern on said displaying means based on said stored image data.

22. The image of claim 21, wherein said pattern is selected by a pattern selecting means for selecting graphic data corresponding to patterns for defining regions of interest from among graphic data comprising stationary phase pattern data stored in a graphic data storing means.

23. The image of claim 22, wherein said pattern selecting means is constituted so as to select predetermined graphical data from among the graphic data stored in the graphic storing means based on coordinate data specified by a cursor means displayed and moveable on the displaying means.

24. The image of claim 21, wherein said stationary phase is selected from the group consisting of a stained polyacrylamide gel and detectable region of a microarray surface.
